(19) United States
(12) Patent Application Publication
(10) Pub. No.: US 2023/0317288 A1
(43) Pub. Date: Oct. 5, 2023

(54) MACHINE LEARNING PREDICTION OF INJECTION FREQUENCY IN PATIENTS WITH MACULAR EDEMA

(71) Applicants: GENENTECH, INC., South San Francisco, CA (US); HOFFMANN-LA ROCHE INC., Little Falls, NJ (US)

(72) Inventors: Yvonna Yun LI, Basel (CH); Verena Lisa STEFFEN, South San Francisco, CA (US); Fethallah BENMANSOUR, Basel (CH); Michel James FRIESENHAHN, South San Francisco, CA (US); Zdenka HASKOVA, South San Francisco, CA (US)

(21) Appl. No.: 18/188,266

(22) Filed: Mar. 22, 2023

Related U.S. Application Data
(63) Continuation of application No. PCT/US2021/051579, filed on Sep. 22, 2021.
(60) Provisional application No. 63/082,256, filed on Sep. 23, 2020.

(51) Int. Cl. G16H 50/20; G16H 20/17

(52) U.S. Cl. CPC G16H 50/20; G16H 20/17

(57) ABSTRACT

A method and system for managing a treatment of a subject diagnosed with a macular edema condition. Subject data for a subject is received. The subject data comprises best corrected visual acuity (BCVA) data for the subject. An input for a computational model is generated using the subject data. An injection frequency for the treatment of the subject diagnosed with the macular edema condition is predicted, via the computational model, based on the input.

### CROSS-REFERENCE TO RELATED APPLICATIONS

This application is a continuation of International Application No. PCT/US2021/051579 filed Sep. 22, 2021, which claims priority to U.S. Provisional Patent Application No. 63/082,256, entitled “Machine Learning Prediction of Injection Frequency in Patients with Macular Edema,” filed Sep. 23, 2020, which are incorporated herein by reference in their entirety.

### FIELD

This description is generally directed towards managing the treatment of macular edema. More specifically, this description provides methods and systems for predicting an injection frequency for the treatment of a subject diagnosed with a macular edema condition associated with retinal vein occlusion using a computational model.

### BACKGROUND

Retinal vein occlusion (RVO) is a retinal vascular disease which threatens vision and can lead to macular edema, macular ischemia, and/or retinal neovascularization. RVO occurs when the flow of blood from the retina is blocked. This blockage is typically due to a. blood dot within a retinal vein and typically occurs where atherosclerotic (thickened and hardened) retinal arteries cross and put pressure on the retinal vein. When a retinal vein is blocked, the drainage of blood from the retina is affected, which may lead to hemorrhage and leakage of fluid from the blocked retinal vein. The most common type of RVO is called BRVO (branch RVO), which occurs when one or more smaller retinal veins are blocked. Central RVO (CRVO) is the blockage of the central retinal vein. The least common type of RVO is hemi-retinal RVO (HRVO), which is diagnosed when half of the retina is affected due to a blockage of two branch veins. The most common sight-threatening complication of RVO is macular edema, which, can cause blurring of vision, distorted vision, or vision loss if left untreated. Macular edema occurs due to blood and fluid leaking into the macula, the part of retina responsible for sharp, clear central vision.

The current standard of care treatments for macular edema due to RVO include intravitreal anti-vascular endothelial growth factor (anti-VEGF) treatments. Such anti-VEGF treatments include, for example, ranibizumab and aflibercept. Long-term treatment regimens may vary widely and may range from continuous monthly injections to as needed (pro re nata, PRN) or treat and extend (TAE) dosing after an initial loading dose period. The frequency of subject monitoring and evaluations conducted over time along with the frequency of injections used to achieve and maintain desired visual outcomes over time may be overly burdensome and may result in undesirable clinical outcomes. Accordingly, it may be desirable to have one or more methods or systems that address one or more of these issues relating to the long-term management of treatment for macular edema.

### SUMMARY

In one or more embodiments, a method is provided for managing a treatment of a subject diagnosed with a macular edema condition. Subject data for a subject is received. The subject data comprises best corrected visual acuity (BCVA) data for the subject. An input for a computational model is generated using the subject data. An injection frequency for the treatment of the subject diagnosed with the macular edema condition is predicted, via the computational model, based on the input.

In one or more embodiments, a method is provided for managing a treatment of a subject diagnosed with a macular edema condition. Subject data for a subject diagnosed with the macular edema condition is received. The subject data comprises best corrected visual acuity (BCVA) data for the subject and at least one of demographic data for the subject or image-derived data for the subject. An input is generated for a computational model using the subject data. An injection frequency for the treatment of the subject diagnosed with the macular edema condition is predicted, via the computational model, based on the input by generating an injection frequency output. A schedule recommended for performing a set of medical evaluations for the subject is generated based on the injection frequency output.

In one or more embodiments, a computer system comprises an injection prediction platform and a computational model that is part of the injection prediction platform. The injection prediction platform is configured to receive subject data for a subject and to generate an input using the subject data. The subject data comprises best corrected visual acuity (BCVA) data for the subject. The computational model is configured to predict an injection frequency for the treatment of the subject diagnosed with the macular edema condition based on the input.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIG. 1 is a block diagram of a treatment management system in accordance with one or more embodiments.

FIG. 2 is a flowchart of a process for managing a treatment of a subject diagnosed with a macular edema condition in accordance with one or more embodiments.

FIG. 3 is a flowchart of a process for training a computational model to predict injection frequency in accordance with one or more embodiments.

FIG. 4 is a table illustrating the performance of three machine learning models in accordance with one or more embodiments.

FIG. 5 is a set of plots illustrating the performance of three machine learning models in accordance with one or more embodiments.

FIG. 6 is a plot illustrating the performance of mean BCVA as a predictor for injection frequency in accordance with one or more embodiments.

FIG. 7 is a block diagram of a computer system in accordance with one or more embodiments.

FIG. 8 is a block diagram of a computer system in accordance with one or more embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

### I. Overview

The ability to predict the injection frequency for a treatment for subjects diagnosed with macular edema (e.g., due to RVO) may improve the overall management of long-term treatments for these subjects. Treatment may include, for example, an anti-vascular endothelial growth factor (anti-VEGF) treatment administered via injection(s) during an initial treatment period and, in some cases, during a management period that begins at some point in time after the initial treatment period. The management period may be a predefined period of time or an “as needed” period of time, where the focus of treatment may be to maintain and/or improve upon the subject's response to treatment during the initial period of time.

The population of patients with macular edema due to RVO is heterogeneous with individual patients requiring different numbers of injections during the management period to achieve and/or maintain desired visual outcomes over time. In other words, heterogeneity among the subjects being treated may lead to variability in the number of injections to be administered during the second treatment course. Currently, the variability in the number of injections that may be needed for subjects may make long-term treatment management of patients with macular edema challenging.

For example, a group of subjects with macular edema under the care of a clinician may need different numbers of treatment injections during the management period. However, with currently available methods and systems, the clinician may be unable to quickly and accurately determine which subjects will need fewer injections and which subjects will need more injections during the management period. Accordingly, the clinician may need to perform regular and frequent evaluations of all of the subjects (e.g., monthly, every other month, etc.) to make decisions regarding the treatment injections. However, monthly visits, which may be a standard in a clinical trial setting, are not always feasible in the real world and represent a burden for patients, caregivers, physicians, and the health care system. For example, the clinician may spend a similar amount of time and resources over the entirety of a given management period evaluating a first subject that may need only 0 or 1 injection when compared with the time and resources spent for a second subject that may need 4 or 5 injections during the same management period.

Thus, a desire exists for methods and systems that enable prediction of an injection frequency for a treatment of a subject with macular edema. The embodiments described herein provide methods and systems for making and using such predictions to improve the long-term treatment management of patients with macular edema. In one or more embodiments, a best corrected visual acuity (BCVA) score is received for a subject. The BCVA score is used to generate an input for a computational model. The computational model is used to analyze the input and predict an injection frequency for the treatment of the subject diagnosed with the macular edema condition based on the input. The injection frequency that is predicted may be for a management period that begins at some point in time after an initial treatment period.

In some embodiments, the computational model includes a machine learning model. The machine learning model may have been previously trained and may include, for example, a logistic regression model.

The prediction may be made by generating, via the computational model, an injection frequency output that indicates whether the injection frequency is predicted to be above a threshold injection frequency (or below a threshold injection frequency). For instance, the injection frequency output may indicate that the injection frequency is above the threshold injection frequency. In other cases, the injection frequency output may indicate that the injection frequency is below a threshold injection frequency. This prediction may be made by generating, via the computational model, an injection frequency output that identifies a frequency category from a plurality of frequency categories for the treatment of the subject (e.g., high frequency category, low frequency category, etc.). The high frequency category may correspond to three (3) or more injections during a management period; the low frequency category may correspond to two (2) or fewer injections during the management period.

Recognizing that BCVA may be a primary indicator for whether a subject will need a high (e.g., ≥3) or low (e.g., ≤2) number of injections over the management period, the computational model (which may include the machine learning model) may be trained using training data that includes a mean BCVA score for each training subject. A “training subject” may include a subject or patient whose data contributes to the training data. The mean BCVA score may be with respect to a period of time associated with the treatment course. For example, this period of time may be 2 months, 3 months, 4 months, 5 months, 6 months, etc. The computational model may be trained to accurately predict injection frequency for a subject using the subject's mean BCVA score corresponding to a selected period of time (e.g., 2 months, 3 months, 4 months, 5 months, 6 months, etc.).

In some embodiments, the input sent into the computational model may include other types of data that may improve the predictive capabilities of the computational model. For example, the input may include BCVA data, image-derived data, demographic data, one or more other types of data, or a combination thereof.

The methods and systems described herein may enable medical professionals (e.g., doctors, nurses, clinicians, etc.) to better manage the overall treatment of a subject during the long-term period. For example, if the injection frequency output generated for a subject predicts a high injection frequency (e.g., ≥3 injections during a management period), a schedule may be generated that indicates the subject should be evaluated more frequently (e.g., monthly). If, however, the injection frequency output generated for the subject predicts a low injection frequency (e.g., ≤2 injections during a defined period), a schedule may be generated that indicates the subject should be evaluated less frequently (e.g., every 2 months, every 3 months, etc.). This type of schedule based on the predicted injection frequency may reduce the overall expense, time, and resources expended in the management of the long-term treatment of subjects with macular edema by the medical professional. Further, predicting injection frequency using the methods and embodiments described herein may reduce the computing resources associated with the scheduling and overall management of long-term treatment of subjects with macular edema.

Still further, such prediction capabilities and scheduling capabilities may improve the subject's experience during long-term treatment management. For example, a subject for which a low injection frequency is predicted can avoid unnecessary visits or evaluations with the medical professional, which will ultimately save time and resources and alleviate the burden on all involved, including the subjects, their caregivers, physicians, and the health care system.

Thus, the methods and systems described herein for predicting injection frequency for the treatment of macular edema during long term treatment management may be useful in various scenarios.

### II. Macular Edema Treatment Management

### II.A. Exemplary Treatment Management System

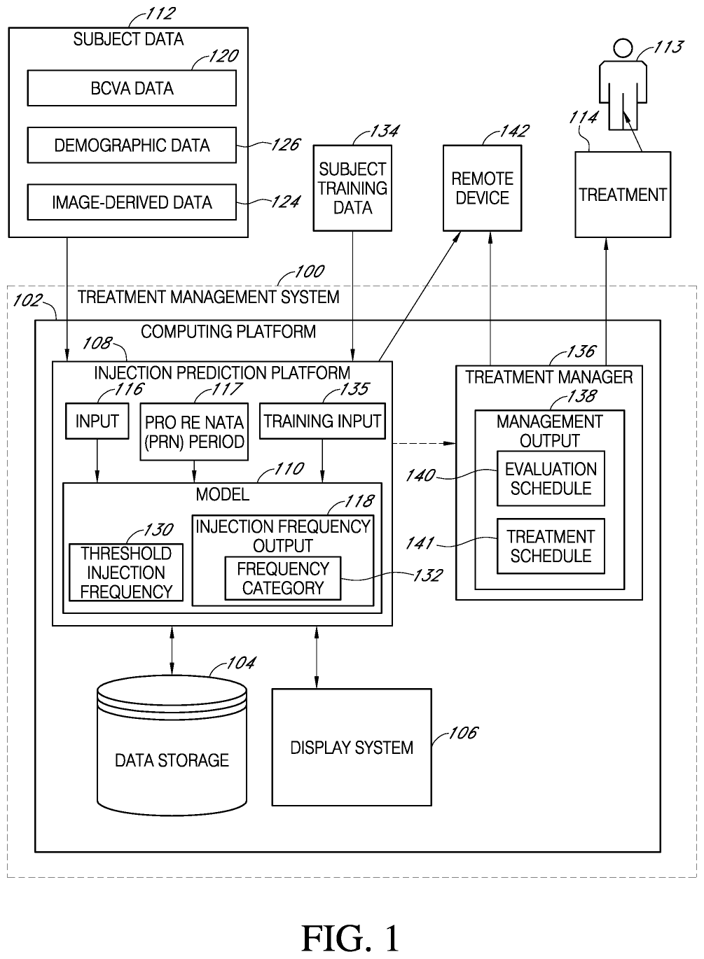

Referring now to the figures, FIG. 1 is a block diagram of a treatment management system 100 in accordance with one or more embodiments. Treatment management system 100 is used to manage the treatment of subjects diagnosed with a macular edema condition associated with retinal vein occlusion (RVO). Treatment management system 100 includes computing platform 102, data storage 104, and display system 106. Computing platform 102 may take various forms. In one or more embodiments, computing platform 102 includes a single computer (or computer system) or multiple computers in communication with each other. In other examples, computing platform 102 takes the form of a cloud computing platform.

Data storage 104 and display system 106 are each in communication with computing platform 102. In some examples, data storage 104, display system 106, or both may be considered part of or otherwise integrated with computing platform 102. Thus, in some examples, computing platform 102, data storage 104, and display system 106 may be separate components in communication with each other, but in other examples, some combination of these components may be integrated together.

Treatment management system 100 includes injection prediction platform 108, which may be implemented using hardware, software, firmware, or a combination thereof. In one or more embodiments, injection prediction platform 108 is implemented in computing platform 102. Injection prediction platform 108 includes injection frequency platform 110. Injection frequency platform 110 may include a computational model that may include any number of models, algorithms, neural networks, equations, functions, or a combination thereof. In one or more embodiments, injection frequency platform 110 includes a computational model that includes at least one machine learning model. In one or more embodiments, the at least one machine learning model may include at least one of a logistic regression model, a deep learning model, a random forest algorithm, a support vector machine (SVM) model, or another type of machine learning model.

In one or more embodiments, injection prediction platform 108 receives subject data 112 for subject 113 that has been diagnosed with a macular edema condition. The macular edema condition may be associated with retinal vein occlusion (RVO) (e.g., central RVO, branch RVO, hemi-retinal RVO). Subject 113 may be, for example, a patient that is undergoing, has undergone, or will undergo a treatment course for treatment 114 for the macular edema condition. Treatment 114 may include, for example, an anti-VEGF treatment that is administered via a number of intravitreal injections, some other type of macular edema treatment that is administered via injection, or a combination thereof. An anti-VEGF treatment may include, for example, ranibizumab, aflibercept, another type of anti-VEGF treatment, or a combination thereof. The treatment course may include a selected number of injections of treatment 114 over a selected period of time. For example, without limitation, the treatment course may include monthly or semi-monthly injections over a selected period of time that is 2 months, 3 months, 4 months, 5 months, 6 months, or some other number of months.

Subject data 112 may be received from a remote device, retrieved from a database, or received in some other manner. In one or more embodiments, subject data 112 is retrieved from data storage 104.

Subject data 112 is used to generate input 116 for a computational model in injection frequency platform 110. The computational model may be trained to use input 116 to predict an injection frequency for treating subjects diagnosed with a macular edema condition during a management period 117. For example, injection frequency platform 110 may receive input 116 and generate, using the computational model, injection frequency output 118 that provides a prediction of injection frequency that is recommended for or expected for subject 113 during the management period 117. The management period 117 may be, for example, a selected period of time following an initial treatment period for treatment 114. The management period 117 may be a predefined period of time such as, for example, 2 months, 3 months, 4 months, 5 months, 6 months, 9 months, 12 months, 2 years, 4 years, or some other period of time following the initial treatment period. In some examples, the management period 117 is an “as needed” or pro re nata (PRN) period of time. The management period 117 may be, for example, an extended dosing period in a treat and extend (TAE) dosing period that follows an initial loading dosing period.

Subject data 112 includes best corrected visual acuity (BCVA) data 120 for subject 113. BCVA data 120 may include, for example, without limitation, a BCVA score for subject 113. This BCVA score may be a mean BCVA corresponding to a selected period of time associated with the treatment course for treatment 114. This selected period of time may be, for example, without limitation, 2 months, 3 months, 4 months, 5 months, 6 months, or some other period of time. For example, the BCVA score may be a mean BCVA over a 3-month period of the treatment course (e.g., baseline to month 3) or a 6-month period of the treatment course (e.g., baseline to month 6). The selected period of time associated with the treatment course may be, in some examples, the same period of time as the period of time for the treatment course.

In some embodiments, BCVA data 120 may include multiple BCVA measurements taken at various points during the treatment course. These multiple BCVA measurements may be transformed into a single BCVA score that forms at least a portion of input 116. For example, the multiple BCVA measurements may be averaged to form a mean BCVA. In other examples, the median of the BCVA measurements may be used as the BCVA score that is used in forming input 116. In one or more embodiments, BCVA data 120 may include a BCVA change.

In one or more embodiments, subject data 112 further includes image-derived data 124 for a set of image-derived parameters, demographic data 126 for a set of demographic parameters, or both. Demographic data 126 may include data regarding, for example, without limitation, age, gender, etc.

Image-derived data 124 may include data derived from one or more images of the retina of subject 113. For example, image-derived data 124 may include data derived from one or more optical coherence tomography (OCT) images, one or more color fundus photography (CFP) images, one or more fluorescein angiography (FA) images, or a combination thereof. This data my include data corresponding to various features associated with the retina and/or optic disc.

Image-derived data 124 may include, for example, without limitation, a central thickness data. The central thickness data may include at least one of central foveal thickness (CFT) data or central subfield thickness (CST) data. In some embodiments, the central thickness data is a mean central thickness corresponding to a selected period of time associated with the treatment course for treatment 114. Central thickness data may be one example of anatomical data.

In some embodiments, the CFT data includes, for example, without limitation, multiple CFT measurements taken at various points during the treatment course. These multiple CFT measurements may be transformed into a single CFT measurement that forms a portion of input 116. For example, the multiple CFT measurements may be averaged to form a mean CFT. In other examples, the median of the CFT measurements may be used as the CFT measurement that is then used in forming input 116.

In some embodiments, the CST data includes, for example, without limitation, multiple CST measurements taken at various points during the treatment course. These multiple CST measurements may be transformed into a single CST measurement that forms a portion of input 116. For example, the multiple CST measurements may be averaged to form a mean CST. In other examples, the median of the CST measurements may be used as the CST measurement that is then used in forming input 116.

Image-derived data 124 may include, for example, without limitation, data for at least one of a parameter corresponding to a presence of a subretinal fluid, a parameter corresponding to a presence of retinal thickening, a parameter corresponding to a presence of a cystoid space within a selected distance of a center of the retina (i.e. the fovea), a parameter corresponding to a presence of an epiretinal membrane (or surface wrinkling), a parameter corresponding to a presence of a pigment disturbance, a parameter corresponding to a presence of collateral vessels on disc, a parameter corresponding to a presence of retinal collateral vessels, a parameter corresponding to a presence of retinal hemorrhage, a total area of leakage in the central subfield, a total area of leakage in the central inner outer subfield, a total area of cyst change in the central subfield, a total area of cyst change in the central inner outer subfield, a treatment scar parameter, or another type of image-derived parameter. The treatment scar parameter may be a parameter that indicates a presence or absence of any scars resulting from treatment (e.g., laser treatment such as focal or grid laser photocoagulation).

The above-described parameters corresponding to a “presence” of a feature (e.g., presence of retinal thickening, presence of subretinal fluid, etc.) may be binary parameters. For example, these parameters may have a value selected from a first value that indicates the presence of the feature and a second value that indicates an absence of the feature. The parameters for “total area of leakage” for the central subfield of the retina and the central inner outer subfield of the retina may be calculated as values with respect to disc area (DA). The disc area may be the area measured for the optic disc. The above-described parameters for “total area of cyst change” for central subfield of the retina and the central inner outer subfield of the retina may be similarly calculated as values with respect to disc area.

The treatment scar parameter may correspond to the presence of one or more scars resulting from a previous treatment for macular edema, such as a previous laser treatment. The previous laser treatment may have been, for example, a laser photocoagulation treatment (e.g., grid laser photocoagulation, focal laser photocoagulation). The treatment scar parameter may be, for example, a binary parameter that indicates the presence or absence of a scar(s) from a previous laser treatment for macular edema.

Input 116 may be formed in various ways. In one or more embodiments, the various types of data included in subject data 112 may be may be combined to form input 116. In other embodiments, some portion or all of subject data 112 may be preprocessed or transformed prior to the combining of this data to form input 116. For example, normalization, one-hot encoding, filtering, and/or other types of preprocessing/transformation operations may be used to form input 116 from subject data 112.

Injection frequency platform 110 receives input 116, analyzes input 116, and generates injection frequency output 118. Injection frequency output 118 provides a prediction of the injection frequency for treatment 114 that is expected for or recommended for subject 113. For example, injection frequency output 118 may provide an indication of whether the injection frequency is predicted to be above threshold injection frequency 130 (or below a threshold injection frequency 130). As one example, injection frequency output 118 may indicate that the predicted injection frequency is above threshold injection frequency 130. As another example, injection frequency output 118 indicates that the predicted injection frequency is below threshold injection frequency 130.

Threshold injection frequency 130 may be, for example, but is not limited to, two (2) injections during the management period 117, three (3) injections during the management period 117, or some other number of injections. Injection frequency output 118 may be, for example, a binary output having two possible values, with one value indicating that the predicted injection frequency is above (or below) the threshold injection frequency 130 and the other value indicating that the predicted injection frequency is not above (or not below) the threshold injection frequency 130. In some embodiments, injection frequency output 118 may be a probability that the injection frequency is above (or below) threshold injection frequency 130. This probability above, for example, 0.5, 0.6, 0.7, 0.8, or some other probability threshold may be considered a prediction that the injection frequency is above (or below) threshold injection frequency 130.

In one or more embodiments, injection frequency output 118 may identify frequency category 132 from a plurality of frequency categories for treatment 114 of subject 113. For example, the frequency categories may include a high frequency category (e.g., ≥3 injections during the management period 117) and a low frequency category (e.g., ≤2 injections during the management period 117). In some examples, the frequency categories include a low frequency category (e.g., ≤2 injections during the management period 117), a moderate frequency category (e.g., 3 or 4 injections during the management period 117), and a high frequency category (e.g., ≥5 injections during the management period 117).

A computational model in injection frequency platform 110 may be trained to generate injection frequency output 118 using subject training data 134. Subject training data 134 may include, for example, training data similar to subject data 113. For example, the training data may include training BCVA data, training image-derived data, and/or training demographic data. Subject training data 134 may include data for a plurality of training subjects (e.g., over 300 training subjects, over 400 training subjects, etc.). Subject training data 134 may include data collected, measured, derived, computed, and/or otherwise obtained for the training subjects over a treatment course (e.g., 6 months) and an observation period following the treatment course (e.g., 6 months).

Subject training data 134 is used to form training input 135 for injection frequency platform 110. In one or more embodiments, subject training data 134 may be preprocessed or transformed prior to the combining of this data to form training input 135. For example, normalization, one-hot encoding, filtering, and/or other types of preprocessing/transformation operations may be used to form training input 135. In one or more embodiments, subject training data 134 may be filtered based on a set of exclusion criteria to form training input 135.

A computational model in injection frequency platform 110 is trained using training input 135 to generate injection frequency output 118 with a level of accuracy that enables injection frequency output 118 to be used in the management of treatment 114 for subject 113 following the treatment course. For example, treatment management system 100 may further include treatment manager 136, which may be implemented using software, hardware, firmware, or a combination thereof. In one or more embodiments, treatment manager 136 is implemented within computing platform 102. Treatment manager 136 may be in communication with injection prediction platform 108. Treatment manager 136 may receive injection frequency output 118 from injection prediction platform 108, process injection frequency output 118, and generate management output 138 for use in managing the long-term treatment of subject 113.

Management output 138 may include, for example, evaluation schedule 140, treatment schedule 141, or both. Evaluation schedule 140 may include a recommended schedule for a medical professional performing a set of medical evaluations of subject 113 based on injection frequency output 118. A medical evaluation may be, for example, a physical evaluation of the vision of subject 113, the retina of subject 113, or both as conducted by a medical professional. When injection frequency output 118 indicates that a higher frequency of injections is expected or recommended during the management period 117 for subject 113, evaluation schedule 140 may recommend that a greater number of medical evaluations be performed for subject 113 during the management period 117 as compared to when injection frequency output 118 indicates a lower frequency of injections that are expected or recommended during the management period 117 for subject 113.

In one or more embodiments, evaluation schedule 140 identifies a number of medical evaluations to be performed, a timing (e.g., a regular interval) for performing the medical evaluations, one or more recommendations regarding the scheduling of the medical evaluations, or a combination thereof. In one or more embodiments, evaluation schedule 140 includes a list of recommended dates for scheduling the medical evaluations.

The medical professional may use evaluation schedule 140 to schedule the medical evaluations for subject 113. At each of these medical evaluations, the medical professional may evaluate subject 113 to determine whether, for example, the visual acuity of subject 113 (e.g., BCVA) warrants another injection of treatment 114.

When a medical professional or clinic oversees many subjects who have macular edema, generating evaluation schedule 140 for each of these subjects may help the medical professional or clinic spend less time and resources in the overall management of the long-term treatment of these subjects. Further, generating evaluation schedule 140 for each of these subjects may help the medical professional or clinic manage injection inventory for treatment 114.

Management output 138 may include treatment schedule 141 that is recommended to be used by the medical professional in treating subject 113 during management period 117. Treatment schedule 141 may include, for example, an identification of a number of injections to be administered, a timing (e.g., a regular interval) for performing the administration of the injections, one or more recommendations regarding the scheduling of the injections, or a combination thereof. The number of times treatment is scheduled within treatment schedule 141 may depend on injection frequency output 118. Treatment schedule 141 is a recommended schedule and the medical professional may choose to modify the actual scheduling of treatments in practice based on medical evaluations performed.

In one or more embodiments, injection frequency output 118, management output 138, or both may be sent to remote device 142 over one or more communications links (e.g., wireless communications links). For example, remote device 142 may be a device or system such as a server, a cloud storage, a cloud computing platform, a mobile device (e.g., mobile phone, tablet, a smartwatch, etc.), some other type of remote device or system, or a combination thereof. For example, management output 138 may be sent to remote device 142 that belongs to a medical professional to aid the medical professional in managing the treating of subject 113. In some embodiments, management output 138 is transmitted in a notification or an email format to a recipient (e.g., medical professional, medical clinic, subject, etc.) that may be viewed on remote device 142.

In one or more embodiments, injection frequency output 118, management output 138, or both may be displayed on display system 106. For example, injection frequency output 118, evaluation schedule 140, or both may be displayed on display system 106 for viewing by a medical professional who can use injection frequency output 118, evaluation schedule 140, or both to determine how to coordinate a set of medical evaluations of subject 113.

In this manner, injection frequency output 118 is used to predict the injection frequency expected for or recommended for treating subject 113 to thereby improve the overall efficiency of managing the long-term treatment of subject 113. Generating evaluation schedule 140 and/or treatment schedule 141 may be one way in which injection frequency output 118 can be used to improve the efficiency of managing the long-term treatment of subject 113. Injection frequency output 118 may be also used in other ways to aid in the long-term treatment management of subject 113.

### II.B. Exemplary Methodologies for Managing Macular Edema Treatment

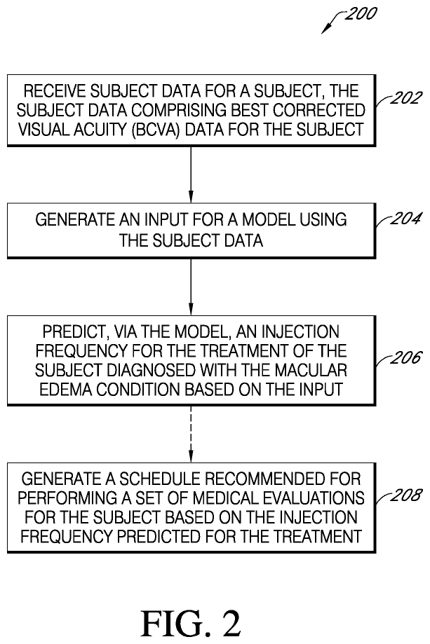

FIG. 2 is a flowchart of a process 200 for managing a treatment of a subject diagnosed with a macular edema condition in accordance with one or more embodiments. In one or more embodiments, process 200 is implemented using the treatment management system 100 described in FIG. 1. For example, process 200 may be used to predict an injection frequency for treatment 114 for subject 113 during the management period 117 in FIG. 1.

Step 202 receiving subject data for a subject, the subject data comprising best corrected visual acuity (BCVA) data for the subject. In step 202, the subject data may take the form of, for example, subject data 112 in FIG. 1. The BCVA data may the form of, for example, BCVA data 120 in FIG. 1. In one or more embodiments, the subject data may be received by injection prediction platform 108 in FIG. 1.

In one or more embodiments, the subject data received in step 202 includes other data. For example, the subject data may further include at least one of image-derived data (e.g., image-derived data 124 in FIG. 1) or demographic data (e.g., demographic data 126 in FIG. 1).

The image-derived data may include, for example, without limitation, data for at least one of a parameter corresponding to a presence of a subretinal fluid, a parameter corresponding to a presence of retinal thickening, a parameter corresponding to a presence of a cystoid space within a selected distance of a center of the retina (i.e. the fovea), a parameter corresponding to a presence of an epiretinal membrane (or surface wrinkling), a parameter corresponding to a presence of a pigment disturbance, a parameter corresponding to a presence of collateral vessels on disc, a parameter corresponding to a presence of retinal collateral vessels, a parameter corresponding to a presence of retinal hemorrhage, a total area of leakage in the central subfield, a total area of leakage in the central inner outer subfield, a total area of cyst change in the central subfield, a total area of cyst change in the central inner outer subfield, a treatment scar parameter, or another type of image-derived parameter. The treatment scar parameter may be a parameter that indicates a presence or absence of any scars resulting from treatment (e.g., laser treatment such as focal or grid laser photocoagulation).

The subject data may include data collected, measured, computed, derived, or otherwise obtained for the subject during a treatment course of a treatment (e.g., treatment 114 in FIG. 1). The treatment may be, for example, an anti-VEGF treatment that is administered via intravitreal injections. The treatment course may be a selected number of injections of the treatment over a selected period of time. The selected period of time may be, for example, 2 months, 3 months, 4 months, 5 months, 6 months, or some other period of time.

Step 204 includes generating an input for a computational model using the subject data. The computational model may be one example of an implementation of a model in injection frequency platform 110 in FIG. 1. The computational model may include, for example, without limitation, a machine learning model. In one or more embodiments, step 204 includes preprocessing or otherwise transforming the subject data to generate the input. For example, one or more preprocessing operations, one or more normalization operations, one or more one-hot encoding operations, one or more linearizing (e.g., converting the categories for a categorical variable into a linear numerical sequence), or a combination thereof may be performed to generate input based on the subject data.

Step 206 includes predicting, via the computational model, an injection frequency for the treatment of the subject diagnosed with the macular edema condition based on the input. The injection frequency is the number of injections expected or recommended for a subject during the management period following the treatment course. The management period may be, for example, without limitation, 2 months, 3 months, 4 months, 5 months, 6 months, a PRN period of time, or some other period of time.

The prediction of the injection frequency ins step 206 may be performed by generating an injection frequency output, such as injection frequency output 118 in FIG. 1. The injection frequency output may indicate whether or not the injection frequency is predicted to be above a threshold injection frequency (or below a threshold injection frequency). The threshold injection frequency may be, for example, threshold injection frequency 130 in FIG. 1. In one or more embodiments, the threshold injection frequency is 2 injections during the management period. In other embodiments, the threshold injection frequency is 3 injections during the management period.

The injection frequency output may identify a frequency category from a plurality of frequency categories for the treatment of the subject. For example, the frequency categories may include a high frequency category and a low frequency category. The high frequency category may correspond to, for example, but is not limited to, 3 or more injections during the management period. The low frequency category may correspond to, for example, but is not limited to, 2 or fewer injections during the management period. In some embodiments, the frequency categories may include a low frequency category (e.g., ≤2 injections during the management period), a moderate frequency category (e.g., 3 or 4 injections during the management period), and a high frequency category (e.g., ≥5 injections during the management period).

In one or more embodiments, process 200 may further include step 208. Step 208 may include generating a schedule recommended for performing a set of medical evaluations for the subject based on the injection frequency predicted for the treatment. For example, the schedule may be generated based on the injection frequency output generated. The schedule may be, for example, evaluation schedule 140 in FIG. 1. The schedule may include, for example, a recommended schedule for medical evaluations of the subject to determine whether an injection of the treatment should be administered to the subject to maintain or improve visual gains. Visual gains may be measured by, for example, without limitation, a number letters of improvement with respect to BCVA as compared to a pre-management period BCVA score (e.g., a baseline BCVA score, the mean BCVA score, or some other BCVA score corresponding to at least a portion of the initial treatment period).

In one or more embodiments, step 208 may be performed using treatment manager 136 in FIG. 1. In some embodiments, step 208 may be performed using the computational model. For example, the computational model may be capable of generating a final schedule output, which includes the schedule, based on the injection frequency output generated by the computational model.

The injection frequency output generated as part of step 206, the schedule generated in step 208, or both may be sent over one or more communications links (e.g., wireless communications links) to one or more remote devices. For example, the schedule may be sent to a server, a cloud storage, a cloud computing platform, a mobile device (e.g., mobile phone, tablet, a smartwatch, etc.), some other type of remote device or system, or a combination thereof. For example, the schedule may be sent to the device or system of a medical professional and/or the device or system of the subject. In some embodiments, the schedule is transmitted in an email format to a recipient (e.g., medical professional, medical clinic, subject, etc.).

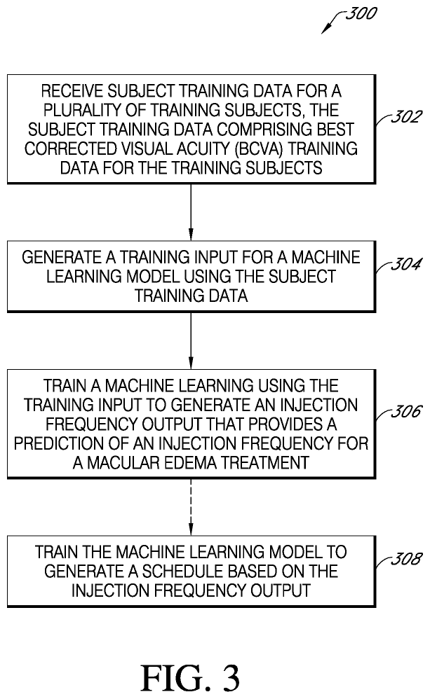

FIG. 3 is a flowchart of a process 300 for training a computational model to predict injection frequency in accordance with one or more embodiments. In one or more embodiments, process 300 is implemented using injection frequency platform 110 described in FIG. 1. For example, process 300 may be used to train a computational model within injection frequency platform 110 in FIG. 1 to predict an injection frequency for a macular edema treatment.

Step 302 receiving subject training data for a plurality of training subjects, the subject training data comprising best corrected visual acuity (BCVA) training data for the training subjects. The subject training data, which may take the form of, for example, subject training data 134 in FIG. 1, may be formed from data generated during one or more clinical trials. The BCVA training data may be similar to, for example, BCVA data 120 in FIG. 1. The BCVA training data may include, for example, without limitation, the mean BCVA scores computed for the training subjects over a period of time corresponding to a treatment course. This period of time may be, for example, 3 months, 6 months, or some other period of time. The training data may further include data regarding the number of injections administered to the training subjects during an observation period following the treatment course to maintain or improve upon the visual gains achieved during the treatment course. The observation period may be, for example, 3 months, 6 months, 9 months, or some other period of time. In one or more embodiments, the observation period may be the same period of time as that of the treatment course.

In one or more embodiments, the subject training data further includes demographic training data, image-derived training data, or a combination thereof. The image-derived training data and the demographic data may be similar to, for example, image-derived data 124 and demographic data 126, respectively, as described with respect to FIG. 1.

Step 304 includes generating a training input for a computational model using the subject training data. The training input may be, for example, training input 135 in FIG. 1. Step 304 may include, for example, performing any number of or combination of preprocessing operations, normalization operations, or one-hot encoding operations. In some embodiments, generating the training input includes filtering the subject training data to exclude the data for certain training subjects. For example, without limitation, the training data may be filtered to exclude subjects who received sham (non-treatment) injections, subjects who did not complete the study for the full duration (the full treatment course plus the full observation period), the subjects who received fewer than a selected number (e.g., 4) of injections during the treatment course, the subjects with certain missing data (e.g., one or more missing image-derived parameters), or a combination thereof.

The computational model may be, for example, one implementation of a computational model in injection frequency platform 110 in FIG. 1. The computational model may include, for example, without limitation, a machine learning model (e.g., a logistic regression model).

Step 306 includes training the computational model using the training input to generate an injection frequency output. The injection frequency output may be, for example, injection frequency output 118 in FIG. 1. Adding image-derived training data, demographic data, or both to the BCVA training data in the subject training data received in step 302 may improve the overall accuracy of the predictions made using the computational model. For example, adding central thickness data may improve the overall accuracy of the predictions made using the computational model. As another example, adding central thickness data and data for one or more other image-derived parameters may improve the overall accuracy of the predictions made using the computational model.

In one or more embodiments, process 300 further includes step 308. Step 308 may include, for example, training the computational model to generate a schedule based on the injection frequency output. The schedule may be, for example, evaluation schedule 140 in FIG. 1.

### III. Exemplary Experiments

### III.A. Methodology

Machine learning models were trained and tested using training data formed from the data of the BRAVO (NCT00486018) and CRUISE (NCT00485836) Phase 3 clinical trials for ranibizumab. The BRAVO study was used to form training data for training subjects diagnosed with branch RVO (BRVO) and those diagnosed with hemi-retinal RVO (HRVO), and the CRUISE study was used to form training data for training subjects diagnosed with central RVO (CRVO).

In both the BRAVO and CRUISE trials, subjects who were given the active treatment (ranibizumab) were given either 0.3 mg or 0.5 mg. The treatment course includes a 6-month treatment period in which monthly injections were given. Following the 6-month treatment course, the subjects were monitored over an observation period of 6 months with monthly medical evaluations to determine whether additional injections of the treatment were needed. This determination was made based on whether the subject's BCVA fell below a predetermined threshold and/or features derived from OCT images of the subject's retina met selected criteria.

Analysis of the clinical trials revealed that the injection frequency over the 6-month management period (following the initial 6 monthly loading period) needed by subjects to maintain the initial vision gained varied between 0 and 6 injections. The initial group of training subjects included a total of 789 subjects from both the BRAVO and CRUISE trials. The training data were filtered using a set of exclusion criteria to form training inputs for the machine learning models. Subjects who received sham (non-treatment) injections, subjects who did not complete the study for the full duration (12 months=the 6-month period of 6 initial monthly loading doses followed by the full 6-month-long variable dosing period with monthly visits), subjects who received fewer than 4 injections during the initial 6-month loading treatment course, and subjects with certain missing data (e.g., one or more missing image-derived parameters) were excluded such that the training input was formed using the training data for 419 subjects.

The training input for a first machine learning model (Model 1) included mean BCVA corresponding to a 3-month period of the treatment course (e.g., baseline to month 3). The training input for a second machine learning model (Model 2) included mean BCVA and mean CFT corresponding to the same 3-month period of the treatment course (e.g., baseline to month 3). The training input for a third machine learning model (Model 3) included mean BCVA and image-derived data for a set of image-derived parameters. The set of image-derived parameters included a parameter corresponding to a presence of a subretinal fluid, a parameter corresponding to a presence of retinal thickening, a parameter corresponding to a presence of a cystoid space within a selected distance of a center of the retina (i.e. the fovea), a parameter corresponding to a presence of an epiretinal membrane (or surface wrinkling), a parameter corresponding to a presence of a pigment disturbance, a parameter corresponding to a presence of collateral vessels on disc, a parameter corresponding to a presence of retinal collateral vessels, a parameter corresponding to a presence of retinal hemorrhage, a total area of leakage in the central subfield, a total area of leakage in the central inner outer subfield, a total area of cyst change in the central subfield, a total area of cyst change in the central inner outer subfield, and a treatment scar parameter.

### III.B. Results

After training, the three machine learning models described above were tested.

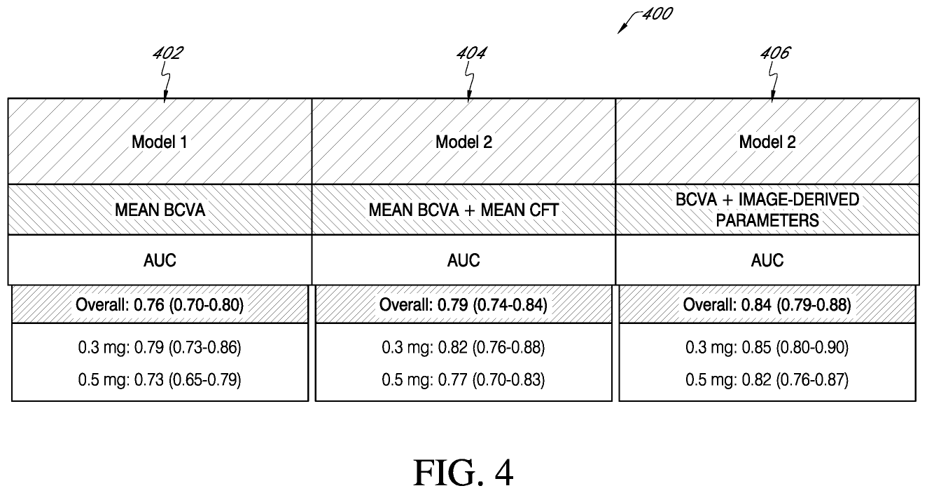

FIG. 4 is a table 400 illustrating the performance of three machine learning models in accordance with one or more embodiments. Column 402 includes performance information for Model 1. Column 404 includes performance information for Model 2. Column 406 includes performance information for Model 3.

Model 1 is a machine learning model that includes a logistic regression model trained to predict an injection frequency using mean BCVA. Model 2 is a machine learning model that includes a logistic regression model trained to predict an injection frequency using mean BCVA and mean CFT. Model 3 is a machine learning model that includes a logistic regression model trained to predict an injection frequency using mean BCVA and image-derived data. Predictive accuracy was assessed for the overall group of training subjects and for each treatment amount group (e.g., 0.3 mg and 0.5 mg) using area under the receiver operating characteristic curve (AUC). As shown in FIG. 4, all three models showed high predictive accuracy, with Model 3 having the highest predictive accuracy.

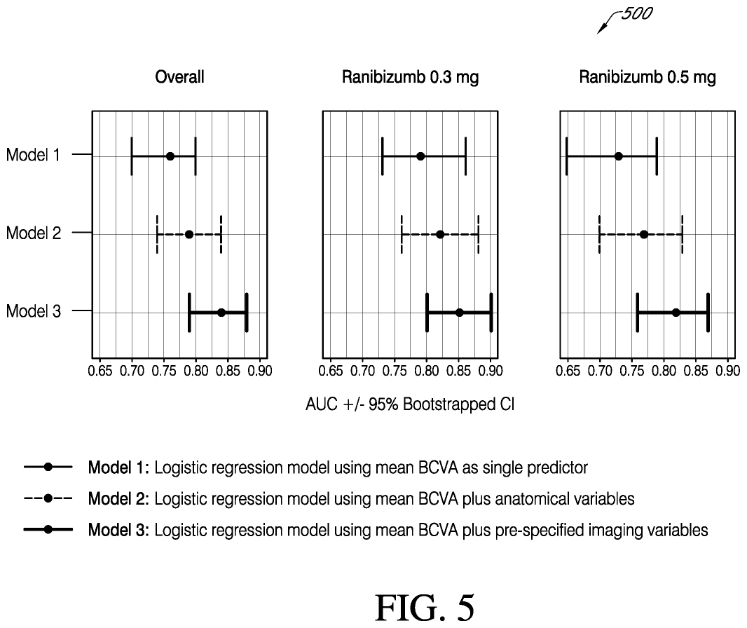

FIG. 5 is a set of plots 500 illustrating the performance of three machine learning models in accordance with one or more embodiments. The performance metrics in FIG. 4 are shown in plot from in FIG. 5. As shown in FIG. 5, the machine learning model that uses both BCVA data and anatomical data (e.g., central thickness data) may have improved performance over the machine learning model that uses BCVA data alone. Further, the machine learning model that uses both BCVA data, anatomical data (e.g., central thickness data), and one or more other image-derived parameters may have improved performance over both the machine learning model that uses BCVA data alone and the machine learning model that uses both BCVA data and anatomical data.

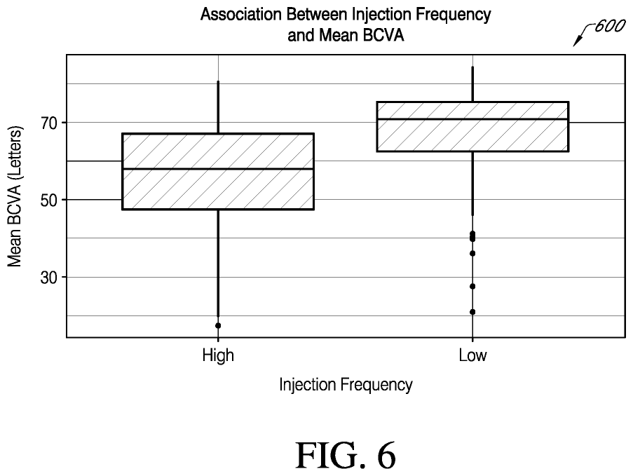

FIG. 6 is a plot 600 illustrating the performance of mean BCVA as a predictor for injection frequency in accordance with one or more embodiments. As shown in plot 600, mean BCVA can be used to distinguish between high and low injection frequencies.

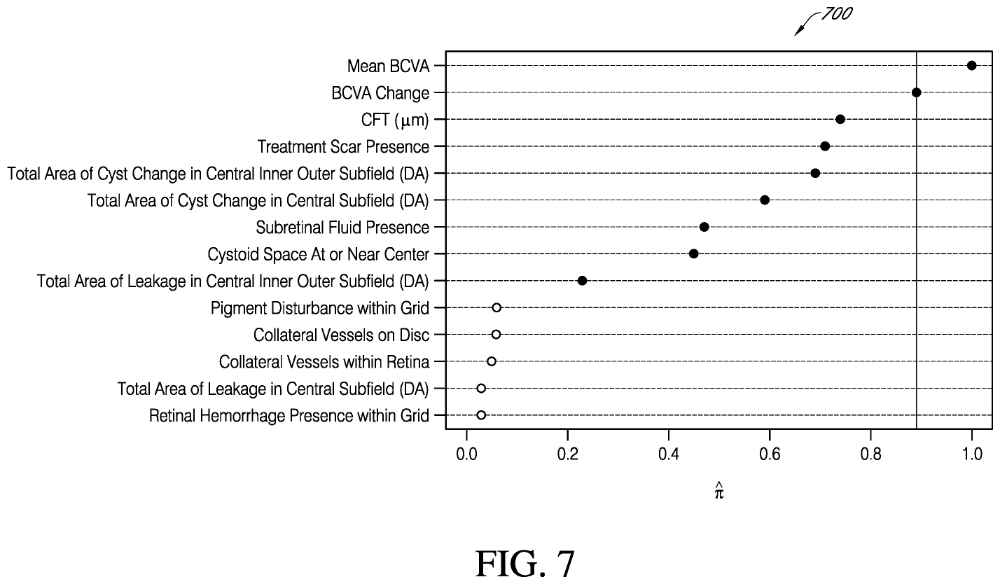

FIG. 7 is a plot 700 showing the relative significance to the predicted output for various parameters in accordance with one or more embodiments. As shown in plot 700, mean BCVA was the most significant parameter.

### IV. Computer Implemented System

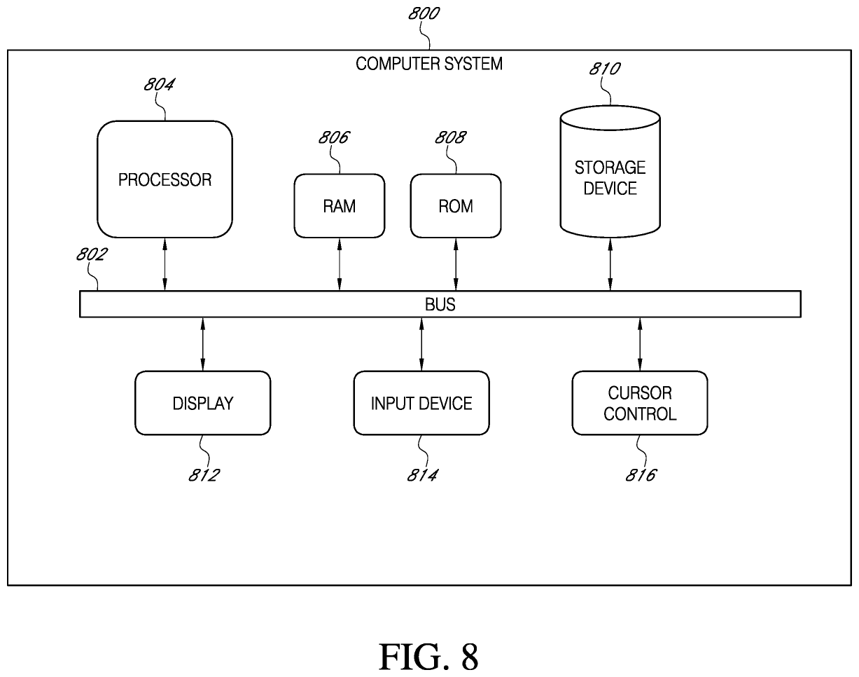

FIG. 8 is a block diagram of a computer system in accordance with one or more embodiments. Computer system 800 may be an example of one implementation for computing platform 102 described above in FIG. 1.

In one or more examples, computer system 800 can include a bus 802 or other communication mechanism for communicating information, and a processor 804 coupled with bus 802 for processing information. In one or more embodiments, computer system 800 can also include a memory, which can be a random-access memory (RAM) 806 or other dynamic storage device, coupled to bus 802 for determining instructions to be executed by processor 804. Memory also can be used for storing temporary variables or other intermediate information during execution of instructions to be executed by processor 804. In one or more embodiments, computer system 800 can further include a read only memory (ROM) 808 or other static storage device coupled to bus 802 for storing static information and instructions for processor 804. A storage device 810, such as a magnetic disk or optical disk, can be provided and coupled to bus 802 for storing information and instructions.

In one or more embodiments, computer system 800 can be coupled via bus 802 to a display 812, such as a cathode ray tube (CRT) or liquid crystal display (LCD), for displaying information to a computer user. An input device 814, including alphanumeric and other keys, can be coupled to bus 802 for communicating information and command selections to processor 804. Another type of user input device is a cursor control 816, such as a mouse, a joystick, a trackball, a gesture input device, a gaze-based input device, or cursor direction keys for communicating direction information and command selections to processor 804 and for controlling cursor movement on display 812. This input device 814 typically has two degrees of freedom in two axes, a first axis (e.g., x) and a second axis (e.g., y), that allows the device to specify positions in a plane. However, it should be understood that input devices 814 allowing for three-dimensional (e.g., x, y, and z) cursor movement are also contemplated herein.

Consistent with certain implementations of the present teachings, results can be provided by computer system 800 in response to processor 804 executing one or more sequences of one or more instructions contained in RAM 806. Such instructions can be read into RAM 806 from another computer-readable medium or computer-readable storage medium, such as storage device 810. Execution of the sequences of instructions contained in RAM 806 can cause processor 804 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 804 for execution. Such a medium can take many forms, including but not limited to, non-volatile media, volatile media, and transmission media Examples of non-volatile media can include, but are not limited to, optical, solid state, magnetic disks, such as storage device 810. Examples of volatile media can include, but are not limited to, RAM 806 (e.g., dynamic RAM (DRAM) and/or static RAM (SRAM)). Examples of transmission media can include, but are not limited to, coaxial cables, copper wire, and fiber optics, including the wires that comprise bus 802.

Additionally, a computer-readable medium may take various forms such as, for example, but not limited to, a floppy disk, a flexible disk, hard disk, magnetic tape, or any other magnetic medium, a CD-ROM, any other optical medium, punch cards, paper tape, any other physical medium with patterns of holes, RAM, PROM, EPROM, EEPROM, FLASH-EPROM, solid-state memory, one or more storage arrays (e.g., flash arrays connected over a storage area network), network attached storage, any other memory chip or cartridge, or any other tangible medium from which a computer can read.

In addition to computer readable medium, instructions or data can be provided as signals on transmission media included in a communications apparatus or system to provide sequences of one or more instructions to processor 804 of computer system 800 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 800 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In one or more embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 800, whereby processor 804 would execute the analyses and determinations provided by these engines, subject to instructions provided by any one of, or a combination of, the memory components RAM 806, ROM, 808, or storage device 810 and user input provided via input device 814.

### V. Exemplary Descriptions of Terms

The disclosure is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, molecular biology, pharmacology, and toxicology are described herein are those well-known and commonly used in the art.

As the terms “on,” “attached to,” “connected to,” “coupled to,” or similar words are used herein, one element (e.g., a component, a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

The term “subject” may refer to a subject of a clinical trial, a person undergoing treatment, a person undergoing anti-cancer therapies, a person being monitored for remission or recovery, a person undergoing a preventative health analysis (e.g., due to their medical history), or any other person or patient of interest. In various cases, “subject” and “patient” may be used interchangeably herein.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one.

As used herein, the term “plurality” may be 2, 3, 4, 5, 6, 7, 8, 9, 10, or more.

As used herein, the term “set of” means one or more. For example, a set of items includes one or more items.

As used herein, the phrase “at least one of,” when used with a list of items, may mean different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, a “model” may include one or more algorithms, one or more mathematical techniques, one or more machine learning algorithms, or a combination thereof.

As used herein, “machine learning” may be the practice of using algorithms to parse data, learn from it, and then make a determination or prediction about something in the world. Machine learning uses algorithms that can learn from data without relying on rules-based programming.

As used herein, an “artificial neural network” or “neural network” (NN) may refer to mathematical algorithms or computational models that mimic an interconnected group of artificial nodes or neurons that processes information based on a connectionistic approach to computation. Neural networks, which may also be referred to as neural nets, can employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters. In one or more embodiments, a reference to a “neural network” may be a reference to one or more neural networks.

A neural network may process information in two ways: when it is being trained it is in training mode and when it puts what it has learned into practice it is in inference (or prediction) mode. Neural networks learn through a feedback process (e.g., backpropagation) which allows the network to adjust the weight factors (modifying its behavior) of the individual nodes in the intermediate hidden layers so that the output matches the outputs of the training data. In other words, a neural network learns by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), or another type of neural network.

As used herein, the term “best corrected visual acuity” may refer to the best visual acuity measurement that can be achieved for a subject via correction (e.g., glasses, contact lenses, etc.).

### VI. Additional Considerations

Any headers and/or subheaders between sections and subsections of this document are included solely for the purpose of improving readability and do not imply that features cannot be combined across sections and subsection. Accordingly, sections and subsections do not describe separate embodiments.

While the present teachings are described in conjunction with various embodiments, it is not intended that the present teachings be limited to such embodiments. On the contrary, the present teachings encompass various alternatives, modifications, and equivalents, as will be appreciated by those of skill in the art. The present description provides preferred exemplary embodiments, and is not intended to limit the scope, applicability, or configuration of the disclosure. Rather, the present description of the preferred exemplary embodiments will provide those skilled in the art with an enabling description for implementing various embodiments. It is understood that various changes may be made in the function and arrangement of elements without departing from the spirit and scope as set forth in the appended claims. Thus, such modifications and variations are considered to be within the scope set forth in the appended claims. Further, the terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

Specific details are given in the present description to provide an understanding of the embodiments. However, it is understood that the embodiments may be practiced without these specific details. For example, systems, processes, and other components may be shown as components in block diagram form in order not to obscure the embodiments in unnecessary detail. In other instances, well-known systems, processes, algorithms, structures, and techniques may be shown without unnecessary detail in order to avoid obscuring the embodiments.

1. A method for managing a treatment of a subject diagnosed with a macular edema condition, the method comprising:
receiving subject data for a subject, the subject data comprising best corrected visual acuity (BCVA) data for the subject;
generating an input for a computational model using the subject data; and
predicting, via the computational model, an injection frequency for the treatment of the subject diagnosed with the macular edema condition based on the input.

2. The method of claim 1, wherein the predicting comprises:
generating, via the computational model, an injection frequency output that indicates the injection frequency as being above a threshold injection frequency.

3. The method of claim 1, wherein the predicting comprises:
generating, via the computational model, an injection frequency output that indicates the injection frequency as being below a threshold injection frequency.

4. The method of claim 3, wherein the threshold injection frequency is two (2) injections during a management period that occurs after an initial treatment period.

5. The method of claim 1, wherein the predicting comprises:
generating, via the computational model, an injection frequency output that identifies a frequency category from a plurality of frequency categories for the treatment of the subject.

6. The method of claim 5, wherein the plurality of frequency categories comprises a high frequency category and a low frequency category.

7. The method of claim 6, wherein the high frequency category corresponds to three (3) or more injections during a management period that occurs after an initial treatment period and wherein the low frequency category corresponds to two (2) or fewer injections during the management period.

8. The method of claim 1, wherein the generating comprises:
generating the input for the computational model using the BCVA data and at least one of image-derived data or demographic data.

9. The method of claim 8, wherein the image-derived data includes central thickness data, wherein the central thickness data comprises at least one of a data for a central foveal thickness (CFT) parameter or a central subfield thickness (CST) parameter.

10. The method of claim 9, wherein the image-derived data comprises data for at least one of a parameter corresponding to a presence of a subretinal fluid, a parameter corresponding to a presence of retinal thickening, a parameter corresponding to a presence of a cystoid space within a selected distance of a center of a retina, a parameter corresponding to a presence of an epiretinal membrane, a parameter corresponding to a presence of a pigment disturbance, a parameter corresponding to a presence of collateral vessels on disc, a parameter corresponding to a presence of retinal collateral vessels, a parameter corresponding to a presence of retinal hemorrhage, a total area of leakage in the central subfield, a total area of leakage in a central inner outer subfield, a total area of cyst change in the central subfield, a total area of cyst change in the central inner outer subfield, or a treatment scar parameter.

11. The method of claim 1, further comprising:
generating a schedule recommended for performing a set of medical evaluations for the subject based on the injection frequency predicted for the treatment.

12. The method of claim 1, wherein the computational model comprises a trained logistic regression model.

13. The method of claim 1, wherein the computational model comprises a machine learning model and further comprising:
training the machine learning model using training data that comprises BCVA training data, wherein the BCVA training data comprises a mean BCVA score for each of a plurality of training subjects corresponding to a selected period of time.

14. A method for managing a treatment of a subject diagnosed with a macular edema condition, the method comprising
receiving subject data for a subject diagnosed with the macular edema condition, the subject data comprising best corrected visual acuity (BCVA) data for the subject and at least one of image-derived data or demographic data for the subject;
generating an input for a computational model using the subject data;
predicting, via the computational model, an injection frequency for the treatment of the subject diagnosed with the macular edema condition based on the input by generating an injection frequency output; and
generating a schedule recommended for performing a set of medical evaluations for the subject based on the injection frequency output.

15. The method of claim 14, wherein the image-derived data comprises central thickness data.

16. The method of claim 14, wherein the image-derived data comprises at least one of a treatment scar parameter, a total area cyst change central subfield, or a total area cyst change central inner outer subfield.

17. The method of claim 14, wherein the computational model comprises a machine learning model.

18. A computer system comprising:
an injection prediction platform configured to receive subject data for a subject and to generate an input using the subject data, wherein the subject data comprises best corrected visual acuity (BCVA) data for the subject; and
a computational model that is part of the injection prediction platform and configured to predict an injection frequency for a treatment of the subject diagnosed with a macular edema condition based on the input.

19. The computer system of claim 18, further comprising:
a treatment manager configured to generate a schedule recommended for performing a set of medical evaluations for the subject based on the injection frequency predicted.

20. The computer system of claim 18, wherein the subject data further comprises data for at least one of a central foveal thickness parameter, a central subfield thickness parameter, a treatment scar parameter, a total area cyst change central subfield, or a total area cyst change central inner outer subfield.

21. (canceled)

22. (canceled)
